(19) United States
(12) Patent Application Publication
(10) Pub. No.: US 2010/0260825 A1
(43) Pub. Date: Oct. 14, 2010

(54) Antifolates for the treatment of cardiovascular, inflammatory, neoplastic, autoimmune and related diseases in sublingual dosage units, film strips, or skin patches

(76) Inventor: Madhavan G. Nair, Mobile, AL (US)

Correspondence Address: MADHAVAN G. NAIR, 7005 CHARLESTON OAKS DRIVE NORTH, P.O. BOX 16144, MOBILE, AL, 36616, US

(21) Appl. No.: 12/386,015

(22) Filed: Apr. 13, 2009

(51) Int. Cl. A61K 31/4985; C07D 475/08; A61P 9/00; A61K 38/13; A61K 39/395; A61K 9/70; A61P 19/02; A61P 17/06; A61P 1/00; A61P 11/06; A61P 25/28; A61P 1/16; A61P 17/00; A61P 37/08; A61P 35/00; A61P 35/02

(52) U.S. Cl. 424/449; 544/260; 514/249; 424/94.1; 514/9; 424/145.1; 424/133.1

(57) ABSTRACT

New metabolism blocked antifolates and their salts are provided along with new indications (coronary heart disease, cardiovascular diseases, stroke and termination of tubal or ectopic pregnancy) and methods (sublingual dosage forms, filmstrips or patches) for their use and delivery. Sublingual dosage forms or trans-dermal patches of metabolically blocked entities of this invention offer superior alternatives to traditional oral dosage forms to achieve greater and predictable therapeutic efficacy, lower toxicity and to overcome drug resistance by virtue of elimination of oxidative deactivation and production of toxic metabolites. Metabolism blocked antifolates bearing a fluorinated benzene ring, a thiophene ring, a pyrrole ring, a furan ring or a 8-deazapteridine ring that are provided in this invention are not previously described. The use of metabolism blocked antifolates to terminate medically complicated pregnancies that are taught herein are new inventions targeted to eliminate drug related toxicity to the host and to enhance therapeutic efficacy.

### FIELD OF INVENTION AND ACKNOWLEDGEMENT

This invention encompasses new metabolism blocked antifolate compounds, new therapeutic indications for their use and methods of delivery to overcome current concerns of toxicity and lack of therapeutic responses. Parts of this invention was supported by National Institutes of Health (U.S.A), grant #CA-27101 and released to the Inventor, M. G. Nair by NIH in a letter dated May 14, 2004 subject to 37 CFR 401.

### DESCRIPTION OF INVENTION

Antifolates are compounds that interfere with tetrahydrofolate utilization [M. G. Nair. “ANTIFOLATES”. In “Cancer Growth and Progression—Cancer Control in Man”. Chapter 10. H. E. Kaiser (Ed). Kluwer Academic Publishers (1990); M. G. Nair. “Chemistry of Antifolates”. In “The Chemistry of Antitumor Agents”. D. E. V. Wilman (Ed) Blackie and Sons (Lond); Chapman and Hall (USA) Chapter—7 (1990)] at various stages of the complex one carbon metabolism mediated by folate coenzymes. Inherently and by mechanisms of action antifolates are very toxic at significantly higher concentrations relative to their therapeutically effective and safe plasma levels. Examples are the well-known antifolate drug Methotrexate (also known as rheumatrex), Tomudex, Alimta and several other antifolate compounds such as Mobiletrex and its analogs that are under development [J. E. Ayling, M. G. Nair, and C. M. Baugh, Editors. “Chemistry and Biology of Pteridines and Folates”. Plenum Publishing Corporation, (1993)]. In fact, although the above compounds are considered safe antifolates, even at their therapeutically effective concentrations folic acid or 5-formyltetrahydrofolate are frequently administered as antidotes to reduce their toxicity in the clinics [A. Amato, M. Fayard, J. Lariccia, J. Mallet, S. Miles and M. G. Nair. “Metabolism-based Antifolate drug design: MDAM and M-Trex”. Pharmacology and Therapeutics; New Millennium. Ed. S. K. Gupta. Pp. 204-212 (2001)]

Chemotherapy of neo-plastic diseases, diseases secondary to uncontrolled or abnormal cell proliferation, inflammatory diseases with or without an auto-immune component using antifolate drugs may utilizes parenteral administration to circumvent fluctuations in plasma drug concentrations secondary to variations in bio-availability when other dosage forms are prescribed (M. G. Nair. Antifolate drugs in Chemotherapy (review). Drug Discovery Today 4: 492-494 (1999). For instance a ten percent variation in bioavailability by the oral route may cause similar fluctuation in plasma drug levels and a ten percent increase relative to the optimal therapeutically effective concentration of the drug in plasma may lead to dangerous and undesired consequences. Since oral administration of antifolates are infrequently used for the most common antifolate indications, methotrexate, also known as rheumatrex is preferred to be administered by the oral dosage form for simplicity and convenience in very low doses for the treatment of rheumatoid arthritis and other auto-immune and inflammatory diseases (G. S. Alarcon, M. O. Castaneda, M. G. Nair, A. Berrocal, E. Paz, W. J. Koopman, and C. L. Krumdieck. Controlled trial of methotrexate versus 10-deazaaminopterin in the treatment of rheumatoid arthritis. Annals. Rheumatic Diseases, 51, 600 (1992); G. Alarcon, O. Castaneda, W. J. Koopman, C. L. Krumdieck, and M. G. Nair. 10-Deazaaminopterin: A New Arthritis Remittive Drug. U.S. Pat. No. 5,030,634 (1991) that require only relatively low doses. Methotrexate usually does not present significant fluctuations in bio-availability during treatment of rheumatoid arthritis due to the very low average dosage of about 15 mg/week combined with its inherently high bio-availability from approximately 28 to 88 percent. [Arthritis and Rheumatism. 60, 1-4 (2009)]. However for potent antifolate drugs with low bioavailability of less than 20 percent even a 5% to 25% variation in absorption among individual patients may either cause dangerous elevation of the drug in plasma with disastrous consequences or lower levels of the drug that are ineffective; clinically manifested as “non-responders”.

It is well documented that some patients with rheumatoid arthritis do not respond to methotrexate [methotrexate non-responders] at doses that were found to be effective in a majority of patients (O. Castaneda and M. Gopal Nair. Controlled trial of methotrexate versus CH-1504 in the treatment of Rheumatoid Arthritis. The J. Rheumatology. 33, 862-64, 2006). It is also known that some patients treated with rheumatrex at normally accepted safe and therapeutically effective doses show undesirable toxicities and significant elevation of liver enzymes, compelling them to withdraw the drug. Although several theories are promulgated to explain the above observations, likely causes may also include sub-optimal and super-optimal plasma drug levels secondary to individual variations in drug absorption from the gut and metabolism during the initial pass, when oral dosage from of the drug is used. Thus taking in to account the above observations of Drs. Castaneda and M. Gopal Nair in at least two clinical trials using methotrexate as a positive standard, it is conceivable for antifolate drugs in general, that non-responsiveness and toxicities might be encountered to a significant extend secondary to fluctuations in plasma drug concentrations by virtue of inter-patient variations in metabolism and bioavailability when administered as oral dosage units.

Absorption of the biological forms of vitamin folic acid present in foods, from human gut is dependent on the levels and activity an enzyme named “conjugase” that is also known as gamma glutamyl hydrolase. Low conjugase levels and activity that are frequently seen in normal humans and in alcoholics may cause folate deficiency. [C. M. Baugh, E. Braverman and M. G. Nair. Biochem. 13:4952 (1974)] Similarly absorption of vitamin B12 from human gut is dependent on the concentration of a protein in the gut known as intrinsic factor. Age related and other variations in intrinsic factor among individuals are well known that may lead to B12 deficiency related abnormality called pernicious anemia causing severe neurological problems and elevation in plasma homocysteine levels. Elevated homocysteine secondary to folate deficiency and B12 deficiency are also well documented and has been recognized as a major risk factor in the incidence of coronary heart disease (CHD) and a number of cardiovascular diseases (CVD). To circumvent folate deficiency and B12 deficiency that are associated with deficiencies of conjugase and intrinsic factor respectively, sublingual administration of these vitamins is being used with considerable success. In fact such sublingual preparations of folic acid and B12 are commercially available to provide sufficient vitamins to the tissues that are independent of the concentrations of conjugase and intrinsic factor in the gut. Thus sublingual administration of folate and B12 can bypass the fluctuations in their bioavailability when the oral dosage forms are used. It is unnecessary to institute intra muscular injections of B12 frequently to overcome B12 deficiency due to mal-absorption. Sublingual administration of folic acid is also preferred to circumvent folate deficiency as a result of mal-absorption when the oral dosage form of the vitamin is used.

The present invention addresses the concerns of inter-patient variability of plasma drug levels, non-responsiveness to therapy and unexpected toxicity using the oral dosage forms by substituting them with sublingual formulations, sublingual strips or films, and by using subcutaneous or trans-dermal patches. The medicated strips may be constructed as single or multi-layer films made of cellulose, starches or other polymeric materials that readily dissolves in mouth facilitating drug delivery to the blood stream via the sublingual artery or buccal mucosa. Effervescing sublingual dosage forms of antifolates using the PEG or sugar or other bases with bicarbonate or tartaric acid (as examples)) may also be used as appropriate to deliver the drugs directly to the blood stream via the sublingual artery.

Metabolism blocked classical antifolates bearing the furan, thiophene or pyrrole ring or with a deaza-pteridine ring, with or without a fluorine-substituted benzene ring at the bridge region are new and novel [FIG. 1 and Calim-1]. These hitherto unknown metabolism blocked antifolates of Claim-1, were designed and methods for their reduction to practice developed, based on mechanistic and stereo-electronic considerations with a view to enhance their folate based enzyme inhibitory potencies.

### PRIOR ART (BACKGROUND OF INVENTION)

Sublingual and other dosage forms [sub lingual tablets, film strips, gel, semisolids and liquids] of antifolates claimed in this Invention are unprecedented and such dosage forms are expected to provide excellent and safe biological responses and therapeutic utility in treating several dozens of diseases that respond to antifolate therapy. No antifolates depicted and recited in FIGS. 1 and 2 (compounds 1 and 2) are currently formulated for clinical use for delivery via the sublingual artery.

This invention encompassing the use of metabolism blocked antifolates 1 and 2 for the prevention, control and treatment of coronary heart disease, cardiovascular disease, stroke and related inflammatory diseases are new and the practice of this invention is expected to have extra ordinary medical utility and commercial success.

This Invention encompassing the utility of compound 1 and 2 for the termination of tubal or ectopic pregnancy and early stage pregnancies that need to be terminated for medical reasons is new and unexpected. Due to the absence of harmful metabolic byproducts of the claimed inventions being formed during therapeutic interventions, the new antifolates claimed herein offer safe and better alternatives of therapies that are currently available for the above medical indications.

This Invention combining the new compounds described in FIG. 1 and Claim-1 and compound 2 with other anti-rheumatic drugs, especially those belonging to the so-called “biologicals” (examples; Humira, Infliximab, Adalimebab, Etanercept, other tumor necrosis factor inhibitors and small molecule anti-metabolites like Arava) are also unprecedented. Likewise, the present Invention claiming the utility of the metabolism blocked antifolate M-Trex [Madhavan G. Nair, U.S. Pat. No. 5,912,251 entitled “Metabolically Inert Anti-inflammatory and Anti-tumor Antifolates”] for the treatment of a variety of diseases encompassing cardiovascular diseases, coronary heart disease and stroke are not only new and novel but also profound and prolific that may have the potential to revolutionize medical interventions of these very serious and often fatal diseases. Termination of early pregnancies using methotrexate with or without the administration of certain prostaglandins is currently available in many countries, but the use of a metabolically blocked antifolate such as M-Trex and other entities claimed in this invention should provide a safer alternative for these indications by virtue of the inability of the claimed compounds including M-Trex to form toxic metabolites during treatment [M. G. Nair, et al. Med. Chem. Research. 9:176-185, 1999].

Sublingual formulations of a number of therapeutic agents and difficultly absorbing vitamins (verapamil, morphine, vitamin B12 and steroids) are currently in use [L. Lea,(www.positivehealth.com/permit/Articles/Colon%20Health/lea13.htm)]. Sublingual formulations of compounds that are relevant but independent to this invention are only very few and they are vitamin derivatives (folic acid and 5-formyltetrahydrofolic acid). Others are certain vitamins that include B12 (cyanocobalamin derivatives) [Survival Enterprises, Idaho, U.S.A No Shot ®.B12, B6 and Folic acid]. Sublingual formulations of antifolates to circumvent problems of bioavailability from oral dosage forms are not described previously. Sublingual dosage forms including but not limited to solids, tablets, gels, semi-solids, liquids or filmstrips and intra-dermal patches of antifolates to ameliorate any diseases that respond to antifolates have no precedence. The concept and reduction to practice of sublingual administration of antifolates to overcome drug resistance, toxicity and to increase patient responsiveness by virtue of creating uniform and predictable antifolate plasma levels is a new invention that, if practiced, is expected have significant and positive therapeutic outcome.

### ADVANTAGES OF THIS INVENTION

This invention teaches a method of administering an antifolate compound directly via the sublingual artery in the mouth (under the tongue and cheeks) that facilitates efficient, direct and quick absorption of the drug to the blood stream bypassing the gastrointestinal (GI) tract. Sublingual administration of drugs and vitamins offers an attractive and efficient alternative for drug delivery in patients who suffer from hyperactive stomach, ulcers, indigestion, celiac disease, stomach cancer, alcoholism and any abnormalities of the GI tract that interferes with absorption. These conditions may also include Crohn's disease, Inflammatory Bowel Disease (IBD) and other inflammatory diseases affecting the GI tract. Sublingual antifolate delivery is predictive of drug blood levels achievable by this route and is independent of any GI tract abnormalities. Sublingual drug delivery stabilizes and increases the bioavailability. No carrier mediated or active transport of the drug is needed when sublingual dosage forms are used to achieve uniform, dose dependent and predictable drug levels in the plasma. The drug is first absorbed directly to the sublingual artery that is connected to the lingual artery and finally enters the blood stream via the common carotid artery. Absorption occurs by passive diffusion and not by active (energy requiring) or carrier mediated transport. It is also likely that absorption of sublingual formulation of the drug may take place by endocytosis or pinocytosis. This method thus overcomes any bioavailability concerns associated with the use of oral dosage units. Often it is only necessary to administer the drug in significantly lower doses by sublingual method relative to the doses required by the oral dosage method as a result of increased bioavailability of the drug via the sublingual route. In fact it has been observed that only 20-25% of estradiol is need to be administered to women by the sublingual route to achieve the same desired bioavailability relative to the dosage used in the oral solid dosage unit of the hormone.

A significant and additional advantage with classical antifolates that include but not limited to methotrexate, aminopterin, alimta, tomudex, 10-DAAM and M-Trex (Mobiletrex) is that they are all acids by virtue of having a glutamate moiety, capable of providing protons to the aqueous chime of the buccal cavity to induce vasodilatation resulting in enhanced absorption of the drug [M. G. Nair. Antifolate drugs in Chemotherapy (review). Drug Discovery Today 4: 492-494 (1999)]. The sublingual absorption of classical antifolates would thus dramatically stabilize plasma drug levels to predictable and uniform concentrations.

Although a number of different types of therapeutic agents, vitamin and minerals are also administered by sublingual delivery interestingly no antifolate compounds are currently administered by this method to treat human disorders that respond to classical antifolate therapies. This may be due to the fact that only methotrexate is currently under use in very low doses to treat chronic diseases such as rheumatoid arthritis and uvitis and very little is known about details of the mechanisms involved in methotrexate unresponsiveness and unexpected toxicity observed in some of the patients. Fluctuations in bioavailability secondary to differences in absorption of the drug from the gut and the resultant significant variations (high or low) in drug plasma levels among patients might account for these observations. Nevertheless, no attempts have yet been made to maintain the bioavailability, predicable and constant by changing the oral formulation of this very important drug, which is also known as rheumatrex. Therefore, sublingual administration of antifolates offers the alternative to address and alleviate the above problems of unresponsiveness and toxicity that are some times very serious secondary to changes in unpredictable drug plasma levels.

This inventions therefore teaches a method to circumvent unpredictable resistance, unresponsiveness and unexpected and some times very severe toxicities associated with the oral administration of antifolate drugs or drug candidates that are used to treat common diseases that are responsive to antifolate therapies.

Besides neoplastic diseases that are characterized by both benign and malignant abnormal cell proliferation, antifolate therapies are useful for the treatment of Inflammatory Diseases (A. Desai, and M. G. Nair. Evaluation of the anti-inflammatory activity and an alternate synthesis of a thiophene substituted 10-deazaaminopterin. Advances in Experimental Medicine and Biology: Chemistry and Biology of Pteridines and Folates. 338:425-428 (1993), Diseases caused by allergens and autoimmune immune diseases (M. G. Nair. Metabolically inert anti-inflammatory and anti-tumor antifolates. U.S. Pat. No. 5,912,251). Although the diseases that respond to antifolate therapies are numerous encompassing a large number of malignant and non-malignant cancers characterized by abnormal cell proliferation, inflammatory diseases, autoimmune diseases, diseases associated with allergic responses, new indications for these compounds are identified in this Invention. The indications that can be treated with either the sublingual administration or by any other preferred route, of novel metabolism blocked antifolates described herein, include, but are not limited to Coronary Heart Disease (CHD), cardiovascular diseases (CVD), stroke, peripheral vascular diseases, termination of ectopic and tubal pregnancies, other abnormal pregnancies, medically defective pregnancies requiring early termination, and termination of early pregnancies associated with any potentially fatal risks for the mother. For pregnancies requiring early termination for medical reasons or any other valid reasons, the antifolate compounds described in this Invention may be administered as such or in combination with other compounds such as misoprostol. The antifolate compound as such or in combination with other compounds may be administered by Parenteral, intraperitonial, intramuscular, sub-cutaneous, sublingual or oral routes. Other methods to administer the drugs are time released and dosage regulated patches, creams, jelly, strips or gels. These time released dosage regulated subcutaneous or trans-dermal patches may be worn over any part of the body that comes in contact with the skin to facilitate absorptive delivery. The creams, jelly, strips or gels containing the antifolate drug may be placed inside a bandage or impregnated to a substance that forms a lining inside the patch. The technical design of the patch or strip may be similar or modifications thereof the currently available patches or strips for therapeutic delivery of steroids, steroid hormones, narcotics, analgesics, birth control medications and others. A list of the abnormalities that may be resolved with the therapeutic use of antifolates described in this invention is provided below.

Inflammatory Diseases Caused by Allergens or Auto-immune Response that Respond to Antifolate Compounds Reported in this Invention:

Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis], Multiple Sclerosis, Ulcerative colitis; Lupus erythematosis, Sjorgen syndrome; Reiter disease; Amyloidosis; Alzheimer's disease; Asthma, Occupational asthma; Exercise induced asthma; Pediatric asthma; Uveitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; Photo toxicity; Serum sickness reaction; Aspirin exacerbated respiratory disease; Hypersensitivity Pneumonitis; Chronic cough; bIsect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; (Connective Tissue disorders): SSc; SLE; PM-DM; Sjogren's; Juvenile chronic arthritis; JIA; Other JIA; Polyarticular JIA; (Hematological): Immune thrombocytopenic purpuria, Evan's; Auto-immune hemolytic anemia; pure red cell; pure white cell and others (Vasculitis): Wegener's; Behcet's; Takayasu; Macroscopic poly. Nodosa; Classical poly. Nodosa; Churg-Strauss; Others (Other Neurological): Mysthenia Gravis and many others described in EBMT Register. [D. Farge, EBMT Autoimmune diseases working party, March 2008. The Rheumatologist. 2: 21, 2008.]

The antifolate drug methotrexate has a bioavailability ranging from 20-80 percent when used as the oral dosage from. The solubility of methotrexate, like other classical antifolates bearing a terminal amino acid moiety is pH dependent and its bioavailability is thus sensitive to the pH variations in the gut of individual patients. Thus if all the patients taking oral dosage form of methotrexate has the ideal and optimal pH in the gut approximately 80 percent of the oral dosage of the drug will be absorbed. If the optimal therapeutic efficacy and response to the disease is based on this theoretical bioavailability, then all of the patients will respond to the therapy. Now if one assumes that one of these patients has a gut pH slightly lower than the optimal to reduce the availability by five percent and assuming that the oral dosage from has 10 mg drug, this patient will have only 7.5 mg of drug absorbed versus the optimal 8.0 mg. Like wise if the pH is slightly higher, then due to enhanced solubility or other factors that results in an increase of Bioavailability by five percent, 8.5 mg of the drug will be absorbed. These minor variations in bioavailability [7.5-8.5 with 8.0 being the optimal] should not detrimentally affect the therapeutic outcome of treatment of the disease with methotrexate in this patient group. Thus if the bio-availability of antifolate is very high using the oral dosage form a 5-10% variations in drug absorption form the gut may not be significant enough to adversely effect the therapeutic outcome. In this example substitution of the oral dosage from with a sublingual dosage form of the drug will not result in significant changes in the therapeutic responses.

### PREFERRED EMBODIMENTS

### Chemical Synthesis: Compound 1

### STEP-1

### 5-Methyl-2-nitrobenzamide

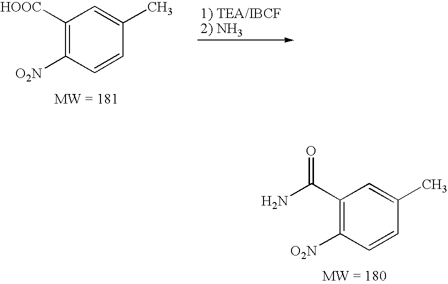

Dissolve (200 mmole, 36.24 grams) of 5-methyl-2-nitrobenzoic acid in 500 mL of dry methylene chloride. After, it was completely dissolved, the reaction was chilled in an ice bath at 0° C. for 20 minutes. Next, (225 mmole, 34.8 mL) triethylamine was added while stirring. After 5 minutes, (225 mmole, 25.4 mL) isobutylchloroformate was added dropwise to the reaction at 0° C. Once it was completely added, the reaction was allowed to stir at room temperature for 2 hours. Finally, anhydrous ammonia was bubbled through the reaction for about 15 minutes until the pH reached 10. The reaction was allowed to stir overnight.

The reaction mixture was evaporated to dryness, added 500 grams of ice, triturated and the resultant precipitate was filtered, washed with distilled water and dried in vacuum. A TLC of the precipitate was taken in 3% or 5% MeOH/CH₂Cl₂ that showed a single spot on exposure to iodine vapors and examination under UV light. Yield, 25.0 g; mp, 176-78° C.; MS, (C₈ H₈ N₂ O₃) calc, 180; found, 180.

### STEP-2

### 5-Methyl-2-nitrobenzonitrile

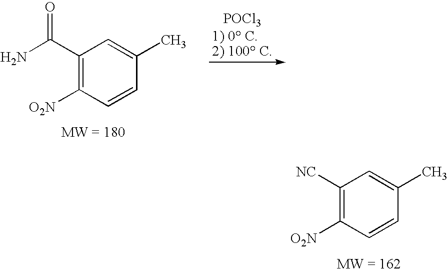

In a 1000 mL three-neck round bottom flask, (0.3 mole, 54.0 grams) of the amide product was dissolved in 300 mL of DMF. The solution was chilled in an ice bath at 0° C. for 20 minutes before (0.32 mole, 30 mL) POCl₃ was added dropwise into the mixture over a period of 30 minutes. The reaction mixture was allowed to stir at room temperature for 40 minutes and then heated in an oil bath at 100° C. for 15 minutes. Once, the flask was cool enough to touch, the reaction mixture was poured over 1 L of ice and the pH was adjusted to 9-10 with ammonium hydroxide. The precipitate thus obtained was washed with water and dried. Upon TLC examination of the product using CH₂Cl₂ it was judged to be pure for further use. Yield, 43.5 g. However minor impurities from the product was removed chromatography on a silica gel column and elution with CH₂Cl₂. Yield, 40.0 g; mp 90-91° C.; MS (C₈ H₆ N₂ O₂) calcd, 162; found, 162.

### STEP-3

### 4′-Carbomethoxy-3-cyano-4-nitrostilbene

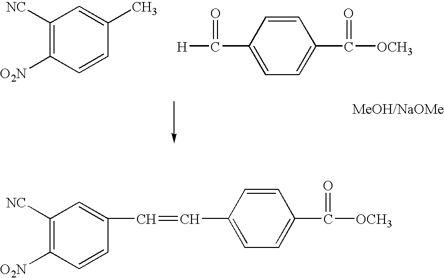

In a 1.0 L round bottomed flask, 12.375 g of the above nitrile and 12.30 grams of methyl-4-formylbenzoate were dissolved in 500 mL MeOH and 20.25 mL NaOMe was added. The reaction is allowed to stir for 96 hours, acidified with TFA to pH 4.0, chilled for 1 hour, filtered and washed with MeOH. Yield, 7.0 g; mp 266-269° C.; MS (FAB), (C₁₇ H₁₂ N₂ O₄), calcd, 308; found 309 (MH+).

### STEP-4

### 4-Amino-3-cyano-4′-methoxycarbonylstilbene

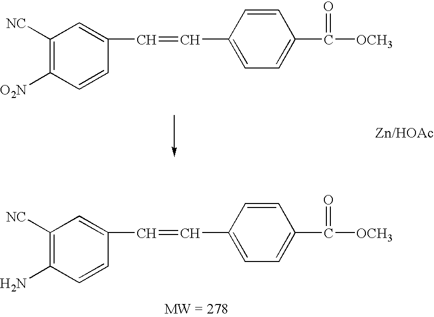

The above stilbene obtained in Step-4 (2.68 grams, 8.7 mmole) was dissolved in 160 mL of glacial acetic acid with stirring. Next, over a period of 15 minutes 32 grams of zinc dust (in four portions of 8 grams each) was added while the stirring continued. After 1 hour, the reaction mixture was filtered, washed successively with glacial acetic acid and methylene chloride, evaporated to dryness, add 25 g of ice, and filtered. The precipitate thus obtained was washed with water and dried in vacuum. The product was re-crystallized using 3:1 MeOH/CH₂Cl₂ (75% MeOH/25% CH₂Cl₂). Yield, 1.8 g, mp, 195-197° C.; MS (FAB), (C₁₇ H₁₄ N₂ O₂), calcd, 278; found 279 (MH+).

### STEP-5A

### Cyclization: 4-Amino-4-deoxy-5,8,10-trideazaethylpteroate (Steps 5-A)

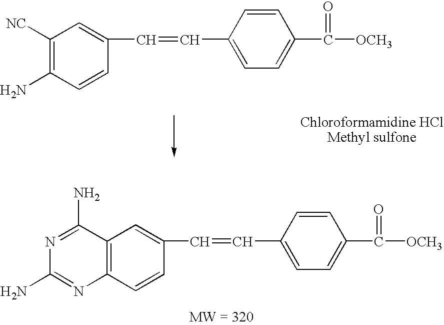

In a 100 mL round bottom flask with a stirring bar, (1.12 grams, 4 mmole) of the amino compound from Step-4, (1.84 grams, 16 mmole) of chloroformamidine HCl, and (7.52 grams, 80 mmole) of methyl sulfone are combined. While stirring, the mixture was heated in an oil bath at 140° C. for 15-20 minutes. After removal from the oil bath the mixture was allowed to cool to 25° C., approximately 70 mL of water was added and then the pH adjusted to 9-10 with ammonium hydroxide. The precipitate thus formed was filtered, washed with copious amounts of water and proceeded to Step-5B. MS (FAB); calcd. (C₁₈ H₁₆ N₄ O₂), 320; found, 321.

### STEP-5B

### Hydrolysis

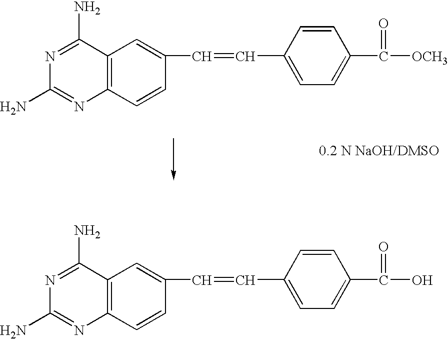

The above crude product was suspended in 400 mL of 0.2 N NaOH and 300 mL of DMSO (added in 75 mL portions). The mixture was allowed to stir overnight at room temperature (approximately 18 hours), chilled the hydrolysate in an ice-bath, and then acidified with 6 N HCl to pH of 7-8 followed by acidification with glacial acetic acid to pH 4. The precipitate thus formed was filtered, washed with water and dried. A small sample of the crude product was dissolve in 5% ammonium hydroxide and subjected to HPLC analysis using a UV monitor at λ=254 nm. The compound was 90% pure and it was used for the next step 6.

### STEP-6

### 4-Amino-4-deoxy-5,8,10-trideazapteroic acid

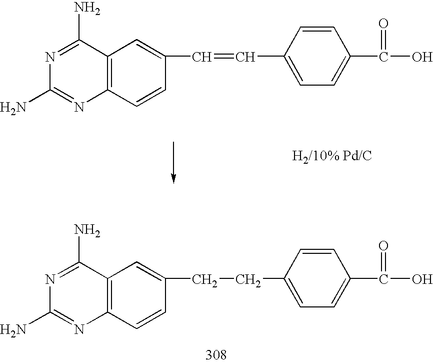

The hydrolysis product was dissolved in 0.2 N NaOH and re-precipitated with 6 N HCl to pH 7-8 then with glacial acetic acid to pH 4. This was to help remove any residual DMSO because it can deactivate the catalyst. The precipitate was washed with water, dissolved in 0.2 N NaOH (50 mL) and hydrogenated at 45 Psig with 500 mg of 10% Pd/carbon as catalyst for 24 hours. The catalyst was filtered out and the filtrate acidified with 6 N HCl to pH 7-8 then with glacial acetic acid to pH 4. The precipitate was filtered, washed with water, dissolved in 5% ammonium hydroxide and applied on a DEAE-cellulose column (25 g). The product was eluted with 15% ammonium hydroxide. On acidification a white precipitate of the desired product was obtained which was washed with water and dried. Examination by HPLC established the purity of this product to be >95%. Yield 900 mg. MS (FAB), calcd. for (C₁₇ H₁₆ N₄ O₂); 308; found, 309 (MH+).

This product was dissolved in 10 mL of TFA, evaporated to dryness and 15 g of ice was added. The precipitate that was formed was filtered, washed with water and freeze-dried for 24 hours before the next step.

### STEP-7

### 4′-Methylene-5,8,10-trideazaaminopterin

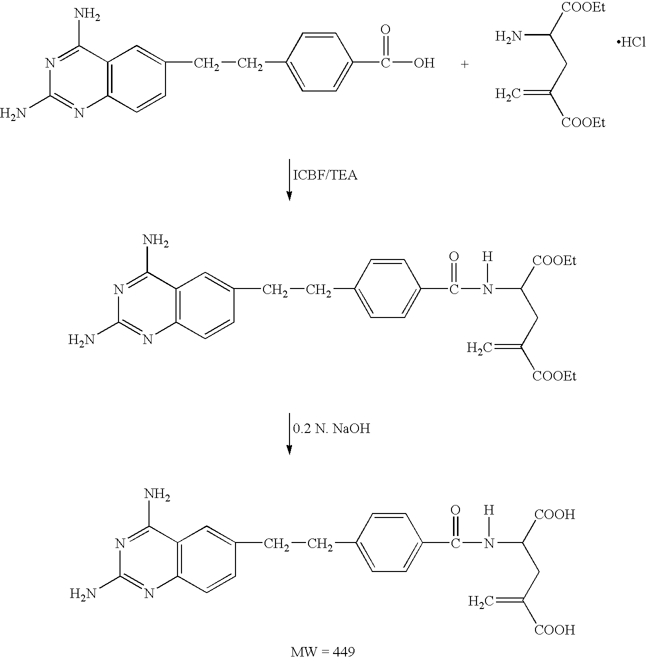

In a 500 mL round-bottomed flask (main reaction flask) 4.23 g of the pteroic acid was dissolved in 200 mL DMF. The flask was chilled in an ice bath at 0° C. for 20 minutes and was added (10 mmole, 1.13 mL) of isobutylchloroformate immediately followed by the addition of (12.5 mole, 1.74 mL) of triethylamine (TEA). The reaction mixture was allowed to stir at room temperature for 1 hour. In a separate flask, (12.5 mole, 3.15 grams) of diethyl-4-methylene glutamate hydrochloride was dissolved in 50 mL DMF, neutralized with (12.5 mmole, 1.74 mL) TEA and it was added to the contents of the main reaction flask. The reaction was allowed to stir overnight and the above coupling procedure was repeated twice. The reaction mixture was then evaporated to dryness and evacuated to remove all DMF. The sample was then hydrolyzed with a mixture of 250 mL of 0.2 N NaOH and 75 mL of acetonitrile overnight under stirring, and acidified to pH 4.0 with glacial acetic acid. The precipitate is filtered, washed with acidified water, dissolved in minimum amount of 5% sodium bicarbonate and applied on a C18 silica gel column made in 12% acetonitrile and water. The column was eluted with 12% acetonitrile: water and the fractions corresponding to >96% purity were combined and acidified to obtain a precipitate. The precipitate was filtered, washed with distilled water and died to obtain the drug product, which was further processed for encapsulation. Yield, 3.25 g, MS (FAB), calcd (C23 H23 N5 O5. H+) 450.1777442; found, 450.175049 (MH+).

In step-3 of the above synthesis when a fluorinated 4-formymethyl benzoate is used the corresponding fluorinated stilbene is formed that can be elaborated to the appropriately substituted fluorinated metabolism blocked antifolate 1 by carrying out the subsequent steps as described in the above synthetic scheme.

If an appropriately substituted thiophene carboxaldehyde is used in step-3 of the above scheme (example 4-carbomethoxy-3-thiophene carboxaldehyde) the corresponding thiophene substituted stilbene is formed that can be elaborated to the thiophene substituted metabolism blocked antifolate depicted in FIG. 1.

Similarly, substitution of 4-carbomethoxy furan-3-carboxaldehyde in Step-3 should yield the corresponding olefin that is further elaborated to the final furan substituted metabolism blocked antifolate depicted in FIG. 1. Thus the above synthetic scheme can be used for the synthesis of all target antifolates described in this invention by simply selecting the appropriate aldehyde in step-3 of the above scheme.

### EXAMPLE-1

Salts of compound 1 or 2

Two major types of slats can be prepared from compound 1 or 2

First, the salts may be formed by the interaction (or reaction) of the carboxylic acid groups with either a strong or weak base. Thus reaction of 1 or 2 with a strong base such as sodium hydroxide or potassium hydroxide yields the corresponding di-sodium or di-potassium salts. If the amount of the strong base is reduced the corresponding mono sodium or mono potassium slats are formed. For practical purposes the di-metallic salts may be preferred. Likewise interaction (or reaction) of 1 or 2 with a weak base such as ammonia or triethylamine will also yield the corresponding di- or mono-amine salts depending on the amount of the weak base used.

Second, the salts of compound 1 or 2 may be formed by interaction of the amino groups with a strong or weak acid. For example treatment of compound 1 or 2 with hydrochloric acid (or hydrogen chloride in an appropriate solvent including water) will yield the corresponding hydrochloride of 1 or 2. Similarly, dissolving 1 or 2 in acetic acid or trifluoroacetic acid and subsequent evaporation of the solvent yields the corresponding acetate of trifluoroacetate salt of 1 or 2.

In a preferred embodiment to make the sodium salt of compound 1 or 2 one molar equivalent of the compound is dissolved in exactly two equivalents of sodium hydroxide solution under stirring. After the dissolution is complete the solution is filtered, concentrated and chilled in the refrigerator till crystallization is complete. The crystals of the di-sodium salt of 1 or 2 are separated by filtration and dried.

In a preferred embodiment the trifluoroacetate salt of compound 1 or 2 is prepared first by dissolving 1 or 2 in excess trifluoroacetic acid under magnetic stirring. When the dissolution is complete the solution is allowed to remain for about 15 minutes and then evaporated to dryness under reduced pressure. The residue is triturated with diethyl ether, filtered and dried.

In another preferred embodiment the hydrochloride of compound 1 or 2 is prepared as follows. One molar equivalent of compound 1 or 2 is suspended in two molar equivalent or an excess of 2-5 (two to five) Normal hydrochloric acid and stirred at room temperature (20 degree Centigrade) till all solid had dissolved. After 30 minutes the solution is filtered to remove minor solid residues if remaining and then evaporated to dryness under reduced pressure. The residue of the hydrochloride of 1 or 2 thus obtained is triturated with diethyl ether, filtered and dried.

### EXAMPLE-2

This group of arthritic patients treated with methotrexate exhibited 20% absorption of methotrexate and the amount of drug in the oral dosage form is tailored to provide optimal therapeutic efficacy and responses calculated from this absorption datum. Since methotrexate is inherently a toxic compound that can cause severe and often unexpected toxic manifestations at significantly higher than optimal therapeutically effective plasma levels treatment of patients exhibiting low bio-availability pose certain significant problems and danger. If 10 mg of the drug is given by the oral route only 2.0 mg is absorbed. If the optimal therapeutic dosage is tailored based on this low absorption, then the therapeutically effective dose without manifestation of significant toxic effects would be an effective accumulation of 2.0 mg of the drug in the plasma. It is reasonable to assume for antifolates that an increase of 50% of drug level in plasma relative to the accepted safe level would lead to disastrous toxic consequences including severe bone marrow depression. Therefore an increase of bioavailability from 20 to 30 percent in this case would effectively increase drug concentration by 50% in the plasma. The 10 percent increase in Bioavailability may well be envisioned by slight pH changes in the gut of patients who may be taking antacids, or medicine to modulate acid secretions, taking other medications or unknown factors. The present invention addresses this very crucial safety issues and provides a means to stabilize drug concentration in plasma to predictable levels by replacing oral dosage form of compound 2 (that is similar to methotrexate) with the sublingual dosage forms, the absorption of which is independent of the variations in biochemical pharmacology or abnormalities of the human gut.

### EXAMPLE-3

Based on ADMEP (Absorption, Distribution, Metabolism, Excretion, Pharmacokinetics) studies conducted with analogous compound 1 in animals and humans during pre-clinical investigations and Phase-I-II clinical trials it was determined that an absorption of 1.0 mg of the drug from the gut to plasma three times a day from divided oral dosage units is optimal (3.0 mg/24 hours) in treating the antifolate responsive disease without causing significant toxicity. It was also determined in this hypothetical study that a concentration of 1.5 mg of the antifolate from the gut to the plasma three times a day (4.5 mg/24 hours) results in unacceptable GI and bone marrow toxicity. The bioavailability of this drug from the oral dosage unit used was only 10 percent as determined from a Phase-I clinical trial in healthy volunteers. Based on the calculated bioavailability figure obtained from healthy humans a solid oral dosage form of 10 mg per dosage unit was formulated to assure optimal plasma drug delivery (1.0 mg×3=3.0 mg/24 hours) to patients. The Bioavailability of this new medication was pH dependent due to solubility reasons and therefore it was possible that fluctuations in bioavailability might occur in certain patients as described in example-2. Thus in certain patients taking antacids or drugs such as certain prostaglandin to reduce acid secretion or other unknown factors the pH of the gut might increase to enhance the solubility and thereby increasing the bio-availability to the 15 to 20% range. Therefore in this particular instance an increase in bioavailability from 10 to 15% might result in dangerous plasma levels of the drug (1.5 mg×3=4.5 mg/24 hours) with unacceptable toxicities and serious side effects.

Likewise a drop in gut pH in certain patients due to various known or unknown reasons can reduce the bioavailability of this drug from 10 to as low as 5% or even lower due to precipitation. This situation may lead to sub-optimal concentration of the drug causing unresponsiveness and ineffective therapy using the 10 mg oral dosage unit.

It is therefore of paramount importance that the plasma levels of this antifolate be kept essentially constant during the entire duration of therapy. As shown in the above examples the required consistency is impossible to achieve using the solid oral unit dosage form of an antifolate drug with low bioavailability in all patients.

The invention of sublingual administration of compound 1 as described herein would circumvent this serious problem as with the use of patches or strips that are also recited and claimed in this invention.

### EXAMPLE-4

### Methotrexate (MTX)-Misadventure:

A clinical pharmacist reviewing a patient's own medications discovered the MTX misadventure. A bottle labeled as ‘2.5 mg methotrexate tablets’ contained actually the 10 mg tablets. The patient had taken six tablets (6×10 mg=60 mg) weekly instead of her usual 15 mg (6×2.5 mg) weekly dose prescribed to treat rheumatoid arthritis. Further investigation revealed that the patient's general practitioner had prescribed MTX 10 mg tablets with the instructions ‘Take one and a half tablets weekly’. Her local pharmacy dispensed the 10 mg tablets and labeled the bottle correctly. Upon arrival at home the patient, out of habit, emptied the contents of the new prescription into an old bottle of MTX tablets which bore the label ‘Methotrexate 2.5 mg tablets. Take 6 tablets weekly’. The patient was unaware of the change in tablet strength and directions, and was subsequently taking the incorrect dose resulting in odynophagia, dysphagia and oesophagitis.

Odynophagia and dysphagia relief was obtained using antacids. Oesophagitis was secondary to MTX. Calcium folinate 15 mg orally four times a day was commenced. Within 24 h the odynophagia had improved and the patient was well enough to be discharged. [S. Yeoh and J. Siderov in the journal Rheumatology. 40: 230-232 (2001)]

Methotrexate misadventure is not a new problem [1-3]. The major risk outlined in these reports relates to frequency of dosing. The dose of MTX used for the treatment of different diseases varies significantly. In the treatment of rheumatoid arthritis and psoriasis, the dose is often once each week, either as a single or divided dose. “There have been instances where this weekly dose has, in error, been taken either daily or on several days each week, resulting in severe unwanted effects, including neutropenia, hepatotoxicity and bone marrow suppression. [1. Brown M A, Corrigan A B. Pancytopenia after accidental overdose of methotrexate. Med. J. Aust. 1991; 155:493-4; 2. Lomaestro, B M; Lesar, T S; Hager T P. Errors in prescribing methotrexate [letter]. J Am Med Assoc 1992;268:2031-2; 3. Isdale, A H; Hordon, L D. Daly M. Methotrexate mishap [letter]. Br J Rheumatol 1994;33:503-4].

Since the compounds claimed in this invention are to be administered also in low doses and more frequently, the methotrexate misadventure type situations may be anticipated during the therapeutic uses of compounds claimed in this invention. The sublingual formulation, antifolate strips and patches recited in this invention therefore are capable of addressing this type of misadventure effectively.

### EXAMPLE-5

In 2002; Castaneda and M. Gopal Nair conducted the clinical trial of a metabolism-blocked antifolate Mobileterx in patients with rheumatoid arthritis to evaluate its efficacy relative to the well-known antifolate drug methotrexate. In this pilot study [O. Castaneda and M. Gopal Nair. [The J. Rheumatology. 33, 862 (2006)] it was observed that as the treatment progressed the plasma concentration of the inflammatory mediator, C-Reactive Protein (CRP) which is also a marker of inflammation consistently decreased.

M-TREX RESPONSES

| | 4 wks | 8 wks | 12 wks | 16 wks |
|---|---|---|---|---|
| Pain | 4.1 | 2.8 | 2.2 | 1.8 |
| HAQ | 1.8 | 1.6 | 1.4 | 1.4 |
| T. Joints | 10.2 | 7.3 | 5.8 | 5.8 |
| S. Joints | 5.6 | 3.5 | 3.0 | 2.9 |
| Pa. GA | 6.0 | 6.0 | 5.0 | 3.0 |
| Ph. GA | 6.2 | 5.0 | 4.5 | 3.0 |
| ESR | 52.7 | 32.7 | 38.3 | 38.6 |
| CRP | 22.1 | 20.7 | 17.7 | 13.6 |

CRP levels are currently correlated with the incidence and progression of cardiovascular diseases (CVD), coronary heart disease (CHD) and stroke. The higher the CRP levels the higher the risk of the incidence of CVD, CHD, Stroke and related diseases. This unexpected and new finding of the ability of the herein disclosed metabolically inert antifolate led to a significant part of the present invention of Compound 1 or 2 as a therapeutic agent for the treatment, prevention, control or reversal of CVD, CHD, Arterioscleroses, Stroke and related diseases in humans and other warm blooded animals. At the present time no antifolates are used as a prophylactic agent for the prevention of the onset or as a therapeutic agent for the control, treatment or reversal of these very serious and fatal inflammatory diseases. Therefore it is realized that the herein disclosed entities 1 and 2 are capable of exhibiting therapeutic properties that address an unmet, but extremely important need, to discover medications to treat and/or prevent a series of serious diseases that have fatal or crippling consequences.

### EXAMPLE-6

### Methotrexate Resistance and Toxicity.

During conventional therapy of rheumatoid arthritis with methotrexate (rheumatrex) in patients, M. Gopal Nair and O. Castaneda observed a significant percentage of methotrexate non-responders up to 40% in certain patient populations. In addition some patients also exhibited significant drug related toxicity including elevation of liver enzymes. In order to unravel the mechanism of methotrexate non-responsiveness M. G. Nair investigated the metabolism of methotrexate and its potential deactivation pathways. This investigation revealed that methotrexate and similar antifolate compounds underwent oxidative deactivation mediated by the enzyme aldehyde oxidase [Cellular Pharmacology. 3; 29-34 (1996)]. Methotrexate was efficiently converted to its corresponding 7-hydroxymethotrexate losing its potent antifolate properties. The amount of aldehyde oxidase vary among patients and those patients with higher levels of this enzyme deactivate the drug efficiently resulting in enhanced methotrexate non-responsiveness relative to those patients with average levels of this enzyme. This oxidative deactivation takes place in significance during the passage of methotrexate through the liver after absorption from the gut. Since the compounds recited in Claims 1 and 2 are metabolically blocked, variations in aldehyde oxidase are independent of plasma levels of the metabolically blocked compounds claimed in this Application.

### EXAMPLE-7

The aldehyde oxidase mediated oxidation of a metabolically inert antifolate compound depicted in FIG. 2 [Calim-2] was investigated by M. Gopal Nair. [Med. Chem. Research. 9:176-185, 1999]. This investigation revealed that the presently disclosed quinazoline and deaza-pteridine based classical antifolates do not undergo or incapable to undergo this oxidative deactivation. These structural modifications built in to the molecular architecture of compounds claimed in this invention effectively and positively eliminate the issues of antifolate non-responsiveness and toxicity secondary to the fluctuating levels of aldehyde oxidase in patients independent of the routes of administration. Thus the oxidative deactivation resistant metabolically blocked antifolates disclosed herein are new inventions that offer significant and substantial therapeutic advantage in the treatment of a number of serious, acute and chronic illness that affects a large number of human beings.

### EXAMPLE-8

Growth Inhibition (GI₅₀ & TGI) of Human
Tumor Cells by 2 and MTX**

| | |
|---|---|
| Log₁₀ GI₅₀ | TGI |

| Cell Line | 2 | 2 | MTX |
|---|---|---|---|
| Leukemia | | | |
| CCRF-CEM | <−8.00 | <−8.00 | −3.77 |
| MOLT-4 | <−8.00 | −7.40 | −3.55 |
| RPMI 8226 | <−8.00 | −7.44 | −3.66 |
| SR | <−8.00 | <−8.00 | −3.50 |
| K562 | <−8.00 | <−8.00 | −3.30 |
| Breast Cancer | | | |
| MCF-7 | <−8.00 | −6.32 | — |
| MCF7-ADR-RES | <−8.00 | −7.26 | — |
| CNS Cancer | | | |
| SF 539 | <−8.00 | −6.3 | −3.66 |

The preceding data summarize the ability of compound 2 to inhibit 50% of the growth of selected tumor cells in culture. The values are expressed as Log₁₀ GI₅₀ values all of which are lower than 10⁻⁸ M. In the same table a comparison is made of the respective values of TGI (Total Growth Inhibition) for 2 and methotrexate.

### EXAMPLE-9

Antitumor Activity and Toxicity** of MTX and Compound 2
[i.v. push Daily × 5 in Nude Mice Bearing Human
HCT-8 Colon Xenograft.]

| Drug¹ (mg/kg/day) | MTGI (%)² | TD (days)³ | T/C % |
|---|---|---|---|
| Control | — | 2.6 ± 0.8 | — |
| M-Trex (4.0 mg/kg/day) | 91.4 ± 2.2 | 16.2 ± 3.2 | 623.08 |
| MTX (1.0 mg/kg/day)³³ | 30.0 ± 9.0 | 4.6 ± 0.6 | 176.92 |

¹The doses used are the Maximum Tolerated doses for each drug on this schedule
²Maximum Tumor Growth Inhibition (% of control)
³Tumor Doubling Time. The above cell culture and tumor xenograft data clearly establish the superiority of the Claimed entities over methotrexate for the treatment of various forms of Cancer.

Advantages of Sublingual dosage units, strips and patches of antifolates:
1. Sublingual dosage units, strips and patches can be used to directly deliver antifolates to the blood stream.
2. Bioavailability of antifolates by sublingual, strips or patches delivery is independent of observed variations in bioavailability of drugs from the gut secondary to GI abnormalities.
3. Bioavailability of antifolates by sublingual dosage forms, strips or patches delivery is independent of variations in bioavailability secondary to pH changes.
4. Bioavailability of antifolates by sublingual dosage forms, strips or patches delivery is independent of variations in bioavailability due to deficiencies of carriers that mediate absorption from the gut.
5. Bioavailability of antifolates by sublingual dosage forms, strips or patches delivery is independent of variations in bioavailability due to unknown and unpredictable variations in the GI tract of individual patients.
6. Bioavailability of antifolates by sublingual dosage forms, strips or patches delivery is independent of variations in bioavailability secondary to differences in the amount of intestinal micro-flora.
7. Bioavailability of antifolates by sublingual dosage forms, strips or patches delivery is independent of variations in bioavailability due to differences in drug metabolism mediated by microorganism in the GI tract.
8. Bioavailability of antifolates by sublingual dosage forms, strips or patches delivery is independent of variations in bioavailability due to changes in enterohepatic circulation.
9. Bioavailability of antifolates by sublingual dosage forms, strips or patches delivery is independent of variations in bioavailability due to instability of the oral dosage unit formulation of the drug in the GI tract.
10. Sublingual dosage forms, strips or patches delivery of antifolates is more convenient for patients with swallowing difficulties.
11. Bioavailability of antifolates by sublingual dosage forms, strips or patches delivery is independent of variations in bioavailability in patients prone to misadventure by swallowing more or less the number of tablets due to forgetfulness.
12. Bioavailability of antifolates by sublingual dosage forms, strips or patches delivery is independent of variations in bioavailability secondary to dietary habits/restrictions/or taking other prescription drugs.
13. Bioavailability of antifolates by sublingual dosage forms, strips or patches delivery is independent of variations in bioavailability due to alcoholism or other related abnormalities.

The antifolate drugs can be formulated for sublingual administration using standard manufacturing procedures. These include sublingual tablets, solids, semi-solids, gels, liquid or strips that are manufactured by existing standard technologies that are available from several Internet websites and published articles and text books on pharmaceutical compounding. Such procedures have been described and any minor variations of such manufacturing procedures may not undermine or may be cited as causative to invalidate or interfere in any way with this Primary Invention; that is “Antifolates for the Treatment of Cardiovascular, Inflammatory, Neoplastic, Autoimmune and Related Diseases in Sublingual Dosage Units, Film Strips, or Skin Patches”.

The antifolate drugs that may be administered using the sublingual dosage units, strips or patches may include but not limited to both classical and non-classical antifolates; antifolates that undergo metabolism (example: hydroxylation, polyglutamylation, hydrolytic cleavage or other bio-transformations known or unknown) or those are metabolically blocked or metabolically inert. Some of the examples may include but are not limited to methotrexate, aminopterin, mobiletrex and several other metabolizable, metabolically stable or metabolically inert classical and non-classical antifolates under development. Any existing or new antifolate drugs that can be used or potentially used in low doses for prolonged periods to treat chronic diseases such as rheumatoid arthritis, psoriasis, prophylactic or therapeutic treatment for coronary heart disease and inflammatory diseases such as asthma may be advantageously formulated for administration by sublingual tablets, solids, semi-solids, gels, liquid, strips or trans-dermal skin patches. In general, diseases that may be treated with sublingual formulations, strips or skin patches of antifolates may include but are not limited to autoimmune diseases, inflammatory diseases and neoplastic diseases. Further, the neoplastic diseases may be comprised of both malignant and benign growths characterized by abnormal cellular proliferation. The antifolates disclosed in this invention may also include non-polyglutamylatable and hydroxylatable antifolates, polyglutamylatable but non-hydroxylatable antifolates, non-polyglutamylatable and non-hydroxylatable antifolates, metabolizable anti-inflammatory and anti-tumor antifolates and metabolically inert anti-inflammatory and anti-tumor antifolates. Sublingual formulations, strips and skin patches of antifolates can also be made using the poly-γ-glutamates of antifolates [M. G. Nair and C. M. Baugh. Biochemistry. 12:3923 (1973)] for therapeutic use.

Sublingual solid or liquid oral dosage forms, strips or skin patches of antifolates may be manufactured by well known existing technologies used by commercial manufacturers. For example the sublingual tablets may be manufactured according the technologies used to manufacture sublingual nitroglycerine or isosorbide dinitrate; the filmstrips may be made by technology similar to the manufacture Listerine strips and skin patches may be made by using technologies similar to deliver testosterone or birth control medications. Antifolates may be formulated as such as the di-acid or as their salts (sodium salt, potassium salt, salts of any mono-valent, divalent (example: calcium) or trivalent metal (example: Aluminum), salts with acids such as hydrochloride, hydrobromide, acetate, trifluoroacetate, tartrate or in general salts with any weak or strong acids. The formulation may be buffered to stabilize the pH with standard buffering agents or combined with citric acid or tartaric acid to provide protons during disintegration of the sublingual dosage unit for vaso-dilation to enhance sublingual absorption. The formulation may be engineered to permit rapid disintegration of the sublingual dosage unit of the antifolate or strips within a few minutes and release of more than 80% of the antifolate to the buccal cavity during disintegration. The antifolate compound may be combined with cellulose, sorbitol, croscamelose-sodium, mannitol, fructose, flavors, gelatin, magnesium stearate, syloid, dicalcium phosphate, xylitol, microcrystalline cellulose and citric acid. One or more disaccharide may be used as fillers. Gelatin may be used as a binder and disintegrants may include but are not limited to sodium starch glycolate, starches and CLPVP. The sublingual formulation may either be a solid dosage form (tablets or strips) or sublingual liquid dosage form consisting of water, alcohol and mannitol, sorbitol or natural honey in different proportions to maximize the absorption. The liquid sublingual dosage form may be used as drops under the tongue for convenient delivery and rapid absorption. The antifolate compounds may include those compounds that inhibit any of the folate-based enzymes of the complex folate metabolic pathways. The above folate based enzymes include a) dihydrofolate reductase (DHFR), b) thymidylate synthase (TS), c) glycinamide Ribonucleotide formyl transferase (GARFT), d) aminoimidazole carboxamide ribonucleotide formyl transferase (AICARFT), e) Serinehydroxymethyl transferase (SHMT), f) folylpoyglutamate synthetase (FPGS) and 10-formyl tetrahydrofolate synthetase.

The terms “L”- and “D”-define the absolute configuration of the methylene glutamate moiety; “S”, “R” and “R, S” are alternate designations to denote the two mirror images (enantiomers) of the antifolate compound having opposite configurations and the racemic mixture having the two enantiomers in equal proportions respectively.

Compound 1 or 2 claimed in this invention can exist as either the D or L enantiomer. The enantiomers are also referred as R or S. Racemic compound 1 or 2 contains equal proportions of D and L [R and S] enantiomers and is devoid of any optical activity. Unequal mixtures of D and L [R and S] enantiomers of compound 1 or 2 may exhibit optical activity (optical rotation), the magnitude and sign (+ or −) of rotation depending on the relative amount of each enantiomer that constitutes the mixture. This invention claims all possible enantiomers and enantiomeric mixtures or diastereomers of compound 1 or compound 2.

Sublingual dosage forms of the widely used antifolate drug methotrexate or any other antifolate drug to overcome bioavailability, toxicity and non-responsiveness concerns have not been previously described. Therefore it is of importance that such formulations of methotrexate also be made available for the clinician for immediate use because of the safety and efficacy benefits claimed in this invention. Since it might take several months or years before the new metabolically blocked antifolates to be developed and marketed to potentially provide alternatives to methotrexate therapy, the necessity of formulating the most prolific and clinically useful antifolate drug methotrexate in is sublingual forms and subcutaneous or intra-dermal patches is of much significance and utility. Due to these important considerations of immediate therapeutic benefits the sublingual dosage forms, consisting of solid, semi-solid, liquid, gel, film strips, along with subcutaneous or trans-dermal patches of methotrexate are included in the claims of this Application. The sub-lingual dosage forms of antifolate drugs claimed in this invention can be manufactured (for example) according to the procedures or modifications of the procedures described in International Journal of Pharmaceutical Compounding (Volume-4, page 129; 2,000) by combining the antifolate drug with sweeteners, (either natural or artificial), suspending and emulsifying agents such as Acacia, flavoring agents to obtain the desired flavor, flavoring oils, and PEG, sugars and other disintegrating polymers as a base. Although there are several sublingual dosage forms of compounds in the market this invention emphasizes on the advantages of the method of sublingual administration of antifolate drugs directly to the blood stream by the sublingual artery and subsequently to the carotid artery to bypass or overcome problematic issues of drug safety, efficacy and toxicities when the oral dosage forms are used.

Pursuant to the preceding disclosures of inventions, I claim:

1. A compound 1 having the following chemical structure or its pharmaceutically acceptable salt; wherein X is not an unsubstituted phenyl ring; 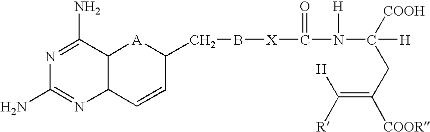
A is selected from a group consisting of the following:
═N—; —NH—; ═C—H—; ═C(CH₃)—; —N(CH₃)—; —O—; —S—. B is selected from a group consisting of the following:
—CH₂—; —CH₂—CH₂—; —C(CH₃)—H; —C(C₂H₅)—H; —NH—; —NCH₃—; —O—; —S—
R′ is selected from the group —H; —CH₃; and —C₂H₅
R″ is selected from the group —H; —CH₃; —C₂H₅; 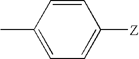
(wherein, Z is an electron-withdrawing group);
X is selected from the group consisting of the following moieties: 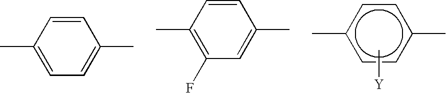
(where, Y is selected from a group consisting of —F, —Cl, —CH₃, —C₂H₅ placed at any of the four positions of the benzene ring by substitution of —H atoms, as mono, di, tri, or tetra substitution). 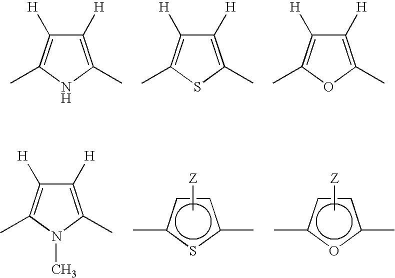
(where, Z is selected from a group consisting of —F, —Cl, —CH₃, or —C₂H₅ placed at any of the two positions of the hetero-cyclic ring by mono substitution or di substitution by replacing one or two hydrogen atoms).

2. A method to treat cardiovascular disease or coronary heart disease or stroke, or for termination of ectopic or tubal or abnormal pregnancy; comprising of administering a compound 2 having the following chemical structure or its pharmaceutically acceptable salt in an amount that is clinically effective and nontoxic to the host. 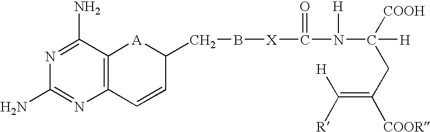
wherein, A is selected from a group consisting of the following:
═N—; —NH—; ═C—H—; ═C(CH₃)—; —N(CH₃)—; —O—; —S— and B is selected from a group consisting of the following:
—CH₂—; —CH₂—CH₂—; —C(CH₃)—H; —C(C₂H₅)—H; —NH—; —NCH₃; —O—; —S—
R′ is selected from the group —H; —CH₃; and —C₂H₅
R″ is selected from the group —H; —CH₃; —C₂H₅; 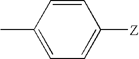
(where Z is an electron-withdrawing group);
X is selected from the group consisting of the following moieties: 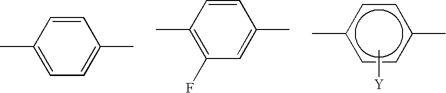
(where, Y is selected from a group consisting of —F, —Cl, —CH₃, —C₂H₅ placed at any of the four positions of the benzene ring by substitution of —H atoms as mono, di, tri, or tetra substitution); 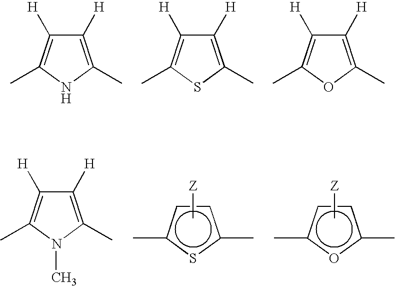
(where, Z is selected from a group consisting of —F, —Cl, —CH₃, or —C₂H₅ placed at any of the two positions of the heterocyclic ring by mono substitution or di substitution by replacing one or two hydrogen atoms).

3. A composition of a compound 1 of Claim-1, or its pharmaceutically acceptable salt comprising of an amount that is therapeutically effective and nontoxic to ameliorate a disease that is selected from a group consisting of: Rheumatoid arthritis; Atherosclerosis; Coronary heart disease; Cardiovascular disease; Stroke; Juvenile rheumatoid arthritis; Psoriasis; Psoriatic arthritis; Crohn's disease; Inflammatory bowel disease; Cystic fibrosis; Sarcoidosis; Ankylosing spondylitis; [Pulmonary fibrosis, Amyloidosis]; Multiple Sclerosis; Ulcerative colitis; Lupus erythematosis; Sjorgen syndrome; Reiter disease; Amyloidosis; Alzheimer's disease; Uvitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; iridocyclitis; Conjunctivitis; Neuromytotonia; Isaacs syndrome; Dyscrasiasis; Atopic dermatitis; Graft vs. Host disease (GVHD); Allograft rejection; Asthma; Exercise induced asthma; Pediatric asthma; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria; Evan's; Auto-immune hemolytic anemia; Vasculitis; Wegener's; Behcet's; Takayasu; Macroscopic poly. Nodosa; Classical poly. Nodosa; Churg-Strauss syndrome; Mysthenia Gravis; Leukemia; Lung cancer; Head and neck cancer; Brain tumor; Lymphoma; Choriocarcinoma; Ovarian cancer; Colon Cancer; Testicular cancer; Kidney cancer; Bladder Cancer; Breast cancer; Liver cancer and Stomach cancer.

4. A pharmaceutically acceptable salt of a compound 2 of claim-2 with a pharmaceutically acceptable carrier.

5. A composition of compound 2 of claim-4 with the addition of a second therapeutic agent selected from a group comprising of leflunomide, a TNF inhibitor, an anti-neoplastic agent, an anti-metabolite, an anti-rheumatic agent, a non-steroidal anti-inflammatory agent, a steroid, misoprostol, a prostaglandin, a statin drug, an ACE inhibitor, a folate coenzyme, an antibiotic, an antiviral agent, a histamine blocker, CDP 870, cyclosporin, humira, infliximab, adalimebab, hydroxychloroquine, sulfasalazine and methotrexate.

6. A sublingual dosage form, filmstrip or skin patch of compound 2 of claim 5 comprising of an amount that is therapeutically effective and nontoxic to ameliorate a disease that is selected from a group consisting of: Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis]; Multiple Sclerosis; Ulcerative colitis; Lupus erythematosis; Sjorgen syndrome; Reiter disease; Amyloidosis; Alzheimer's disease; Asthma; Occupational asthma; Exercise induced asthma; pediatric asthma; Uveitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria; Evan's; Auto-immune hemolytic anemia; Vasculitis, Wegener's; Behcet's; Takayasu; Macroscopic poly. Nodosa; Classical poly. Nodosa; Churg-Strauss syndrome; Mysthenia Gravis; Leukemia; Lung cancer; Head and neck cancer; Brain tumor; Lymphoma; Choriocarcinoma; Ovarian cancer; Colon Cancer; Testicular cancer; Kidney cancer; Bladder Cancer; Breast cancer; Liver cancer; Stomach cancer; Atherosclerosis; Coronary heart disease; Cardiovascular disease and stroke.

7. A composition of a compound 1 or its pharmaceutically acceptable salt according to claim 5 that is administered by the parenteral route, or oral route or sublingual route or by a film strip or by a skin patch comprising of an amount that is therapeutically effective and nontoxic to ameliorate a disease that is selected from a group consisting of Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis], Multiple Sclerosis, Ulcerative colitis; Lupus erythematosis, Sjorgen syndrome; Reiter disease; Amyloidosis; Alzheimer's disease; Asthma; Occupational asthma; Exercise induced asthma; pediatric asthma; Uveitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria, Evan's; Auto-immune hemolytic anemia; Vasculitis; Wegener's; Behcet's; Takayasu; Macroscopic poly. Nodosa; Classical poly. Nodosa; Churg-Strauss syndrome; Mysthenia Gravis; Leukemia; Lung cancer; Head and neck cancer; Brain tumor; Lymphoma; Choriocarcinoma; Ovarian cancer; Colon Cancer; Testicular cancer; Kidney cancer; Bladder Cancer; Breast cancer; Liver cancer; Stomach cancer; Atherosclerosis; Coronary heart disease; Cardiovascular disease and stroke.

8. A sublingual solid, semi-solid, liquid or gel dosage form or strip or skin patch of a compound 2 of claim-2 comprising of an amount that is therapeutically effective and nontoxic to ameliorate, reverse or prevent cardiovascular disease or coronary heart disease or stroke in humans or a warm blooded animal.

9. A method of administration of a compound 2 of claim-2, comprising of an amount that is safe and therapeutically effective to the host but toxic enough to the fetus in combination with or followed by the administration of misoprostol to terminate ectopic, tubal, abnormal or unwanted pregnancy in women or a warm blooded animal.

10. A compound 1 of claim-1, having the “L” (R or S) configuration at the 4′-methylenegltamate moiety.

11. A compound 1 of claim-l, having the “D” (R or S) configuration at the 4′-methylenegltamate moiety.

12. Racemic mixture of a compound 1 or 2 of claim 1 or claim-2, having equal proportions of “L” and “D” (R and S) enantiomers that does not exhibit either Levo or Dextro optical rotation under plane polarized light.

13. A mixture of D and L (R and S) enantiomers of a compound 2 of claim-2, in all possible combinations.

14. A diastereomer or enantiomer of a compound 1 of claim-1.

15. A method to treat by administering sublingually or parenterally, a compound 1 of claim-1, a therapeutically effective and non-toxic amount to ameliorate a disease selected from the group consisting of Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis]; Multiple Sclerosis; Ulcerative colitis; Lupus erythematosis, Sjorgen syndrome; Reiter disease; Amyloidosis; Alzheimer's disease; Asthma, Occupational asthma; Exercise induced asthma; pediatric asthma; Uveitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria, Evan's; Auto-immune hemolytic anemia; Vasculitis, Wegener's; Behcet's; Takayasu; Macroscopic poly. Nodosa; Classical poly. Nodosa; Churg-Strauss syndrome; Mysthenia Gravis; Leukemia; Lung cancer; Head and neck cancer; Brain tumor; Lymphoma; Choriocarcinoma; Ovarian cancer; Colon Cancer; Testicular cancer; Kidney cancer; Bladder Cancer; Breast cancer; Liver cancer; Stomach cancer; Atherosclerosis; Coronary heart disease; Cardiovascular disease and stroke.

16. A sublingual solid, gel, liquid or semi-solid dosage form or film strip or trans-dermal patch of either the L enantiomer or the D enantiomer or a combination of both the L and D (R and S) enantiomers in all possible proportions of a compound 2 of claim-2, comprising of an amount that is therapeutically effective and nontoxic to ameliorate a disease selected from the group consisting of the Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis], Multiple Sclerosis, Ulcerative colitis; Lupus erythematosis; Sjorgen syndrome; Reiter disease; Amyloidosis; Alzheimer's disease; Asthma, Occupational asthma; Exercise induced asthma; pediatric asthma; Uveitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria, Evan's; Auto-immune hemolytic anemia; Vasculitis; Wegener's; Behcet's; Takayasu; Macroscopic poly. Nodosa; Classical poly. Nodosa; Churg-Strauss syndrome; Mysthenia Gravis; Leukemia; Lung cancer; Head and neck cancer; Brain tumor; Lymphoma; Choriocarcinoma; Ovarian cancer; Colon Cancer; Testicular cancer; Kidney cancer; Bladder Cancer; Breast cancer; Liver cancer; Stomach cancer; Atherosclerosis; Coronary heart disease; Cardiovascular disease and stroke.

17. A sublingual gel, liquid, solid or semisolid dosage form or film strip or skin patch of an antifolate compound 2 of claim-2, comprising of an amount in the range of 0.01 milligram to 3,000 mg per dosage unit that is therapeutically effective to treat a disease that responds to methotrexate.

18. The metallic salt of a compound 2 of claim-2, selected from the group of metals consisting of; sodium, potassium, calcium, magnesium, cesium, rubidium, gold, platinum, silver and tin.

19. The organic salt of a compound 2 of claim-2, formed by reaction with a reactant selected from the group consisting of ammonia, aniline, methylamine, ethylamine, diethylamine, dimethylamine, triethylamine, trimethylamine, benzylamine, ethanolamine, diethanolamine, triethanolamine, piperazine, pyridine, piperidine, quinoline, trifluoroacetic acid, acetic acid, flouroacetic acid, bromoacetic acid, hydrogen chloride, hydrogen bromide, hydrogen iodide, hydrogen fluoride, sulfuric acid, nitric acid, tartaric acid, citric acid and phosphoric acid.

20. The metallic salt of a compound 2 of claim-2, formed by reaction with a strong or weak metallic base.

21. The salt of a compound 2 claim-2, formed by reaction with a strong acid or a weak acid

22. A patch or film impregnated or embedded with a compound 2 of claim-2, designed for subcutaneous delivery that is worn over the bare skin or that is able to dissolve in the mouth of a human being or a warm blooded animal.

23. A sublingual gel, liquid, solid, semisolid dosage form or film strip or oral dosage unit or parenteral dosage unit of a compound 1 of claim-1, or its pharmaceutically acceptable salt having the methyleneglutamate moiety possessing either the “L” configuration or “D” configuration, that is administered in a therapeutically effective and non-toxic amount to ameliorate a disease selected from the group consisting of Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis], Multiple Sclerosis, Ulcerative colitis; Lupus erythematosis, Sjorgen syndrome; Reiter disease; Amyloidosis; Alzheimer's disease; Asthma; Occupational asthma; Exercise induced asthma; pediatric asthma; Uveitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria; Evan's; Auto-immune hemolytic anemia; Vasculitis; Wegener's; Behcet's; Takayasu; Macroscopic poly. Nodosa; Classical poly. Nodosa; Churg-Strauss syndrome; Mysthenia Gravis; Leukemia; Lung cancer; Head and neck cancer; Brain tumor; Lymphoma; Choriocarcinoma; Ovarian cancer; Colon Cancer; Testicular cancer; Kidney cancer; Bladder Cancer; Breast cancer; Liver cancer; Stomach cancer; Atherosclerosis; Coronary heart disease; Cardiovascular disease and stroke.

24. A method to treat using a sublingual gel, liquid, solid, semisolid or film strip dosage form or oral dosage unit or parenteral dosage unit or trans-dermal skin patch of a pharmaceutically acceptable salt of a compound 2 of claim-2, having the methyleneglutamate moiety possessing either the “L” (R or S) configuration or the “D” (R or S) configuration, a therapeutically effective and non-toxic amount to ameliorate a disease selected from the group consisting of Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis]; Multiple Sclerosis, Ulcerative colitis; Lupus erythematosis; Sjorgen syndrome; Reiter disease; Amyloidosis; Alzheimer's disease; Asthma; Occupational asthma; Exercise induced asthma; pediatric asthma; Uvitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria, Evan's; Auto-immune hemolytic anemia; Vasculitis, Wegener's; Behcet's; Takayasu; Macroscopic poly. Nodosa; Classical poly. Nodosa; Churg-Strauss syndrome; Mysthenia Gravis; Leukemia, Lung cancer; Head and neck cancer; Brain tumor; Lymphoma, Choriocarcinoma; Ovarian cancer,; Colon Cancer; Testicular cancer; Kidney cancer; Bladder Cancer; Breast cancer; Liver cancer; Stomach cancer; Atherosclerosis; Coronary heart disease; Cardiovascular disease and stroke.

25. A sublingual tablet, solid, liquid, semi-solid, gel, or filmstrip or subcutaneous or trans-dermal skin patch of methotrexate having the following chemical structure, 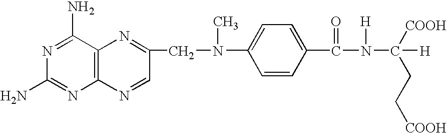
or its pharmaceutically acceptable salt, comprising of an amount that is therapeutically effective and nontoxic to ameliorate a disease that is selected from a group consisting of: Rheumatoid arthritis; Atherosclerosis; Coronary heart disease; Cardiovascular disease; Stroke; Juvenile rheumatoid arthritis; Psoriasis; Psoriatic arthritis; Crohn's disease; Inflammatory bowel disease; Cystic fibrosis; Sarcoidosis; Ankylosing spondylitis; [Pulmonary fibrosis; Amyloidosis]; Multiple Sclerosis; Ulcerative colitis; Lupus erythematosis; Sjorgen syndrome; Reiter disease; Amyloidosis; Alzheimer's disease; Uvitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Asthma; Exercise induced asthma; Pediatric asthma; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Immune thrombocytopenic purpuria; Evan's; Auto-immune hemolytic anemia; Vasculitis; Wegener's; Behcet's; Takayasu; Macroscopic poly. Nodosa; Classical poly. Nodosa; Churg-Strauss syndrome; Mysthenia Gravis; Leukemia; Lung cancer; Head and neck cancer; Brain tumor; Lymphoma; Choriocarcinoma; Ovarian cancer; Colon Cancer; Testicular cancer; Kidney cancer; Bladder Cancer; Breast cancer; Liver cancer and Stomach cancer.
